(19) United States
(12) Patent Application Publication
(10) Pub. No.: US 2014/0121569 A1
(43) Pub. Date: May 1, 2014

(54) ULTRASONICALLY HEATED PROBE

(71) Applicant: Solta Medical, Inc., Hayward, CA (US)

(72) Inventors: Mark E. Schafer, Lower Gwynedd, PA (US); Kelly Ammann, Boulder, CO (US); Kevin J. Hight, Louisville, CO (US)

(73) Assignee: Solta Medical, Inc., Hayward, CA (US)

(21) Appl. No.: 13/835,050

(22) Filed: Mar. 15, 2013

Related U.S. Application Data
(60) Provisional application No. 61/718,580, filed on Oct. 25, 2012.

(51) Int. Cl. A61N 7/02

(52) U.S. Cl. CPC A61N 7/02
USPC 601/3

(57) ABSTRACT

A surgical device includes a probe having a proximate end configured to connect to an ultrasonic driver assembly that generates ultrasonic vibrational energy, and a shaft for conducting the ultrasonic vibrational energy from the proximate end to a distal end. The device also includes a canula located at least partially over the distal end of the probe. A material is interposed between the probe and the canula for converting the ultrasonic vibrational energy into heat energy.

### CROSS-REFERENCE TO RELATED APPLICATIONS

This application claims the benefit of U.S. Provisional Patent Application No. 61/718,580, entitled “ULTRASONICALLY HEATED PROBE,” filed on Oct. 25, 2012, the entire disclosure of which is hereby incorporated herein by reference.

### INTRODUCTION

As people age, there may be an increase in skin tissue laxity, leading to wrinkles and sagging skin. These are not desirable aesthetic conditions. Aesthetic and cosmetic surgeons and dermatologists have tried to remedy the issue using various treatments, including surgical alterations (e.g. face lifts), topical chemical treatments, radiofrequency heating, laser ablation, resistive heating, focal ultrasound, and combinations thereof. It is also often the case that there is a desire to have some skin tightening in conjunction with liposuction, or fat removal. In this case, the removal of the underlying fat can cause the overlying skin to appear saggy. The patient may desire to have both the improved shape resultant from the liposuction procedure, as well as a firmer appearance of the skin. These two desires are often in conflict in practice.

Body tissue includes both Type I (bone, tendon, skin, and other connective tissues) and Type III (muscle, elastic tissues, and collagen tissues). For example, a tissue component that can be made to shrink is collagen. When collagen fibers are subjected to heating, their highly oriented structure is denatured or transformed into an amorphous state, with the net result of tissue shrinkage. In addition to heating, collagen may be denatured chemically, or through a combination of chemical and energy deposition.

There have been a number of energy-based attempts to denature collagen in order to induce a tightening of the skin. Energy-based treatment approaches attempt to apply controlled heating of tissue at a location of interest, which typically denatures the collagen matrix. When collagen denatures, it shrinks, thus causing a tightening of the tissue. If the heating is sufficiently extensive or intense, tissue necrosis occurs, which leads to a wound healing response. During this response, the body absorbs the dead cellular material, the tissue contracts, and a “remodeling” stage occurs in which the dead tissue is replaced with a scar-like matrix. This wound healing response also causes a “tightening.”

There are problems with the current approaches, however. The radiofrequency (“RF”) approach can be applied externally, but the energy density required to cause contraction of the underlying collagen matrix may also cause damage to the overlying skin. A similar issue occurs with laser treatment. While an externally focused ultrasound approach does cause tissue necrosis, remodeling, and contraction, it is limited to a relatively small area such as the eyebrows, and cannot be used over a large area, for instance, over the buttocks. Other approaches involve very controlled heating, but may require multiple incisions, multiple internal heat sources, external cooling devices, etc.

Another approach is the use of ultrasound to dislodge the fat cells prior to aspiration. This approach is best embodied by the VASER® Ultrasound Assisted Liposuction (UAL) system, available from Sound Surgical Technologies LLC, of Louisville, Colo. The VASER system uses ultrasound at a frequency, tip excursion, and application approach that is designed to minimize tissue heating. In liposuction surgery, where the goal is to maximize the safe removal of fat tissue, heating is generally avoided as an unwanted potential complication. This allows the surgeon to concentrate on creating the most efficient and aesthetically pleasing liposuction result, without the need to worry about inadvertently destroying tissue. However, once the liposuction is completed, there is often the desire to induce some level of skin contraction. Because of this, surgeons will sometimes follow a VASER lipoaspiration procedure (or a standard lipoaspiration procedure or a power-assisted lipoaspiration procedure) with the subcutaneous application of a laser fiber. In this case, a high intensity laser is fired down a fiber to create subcutaneous heating. This two-step approach is cumbersome, requiring two completely different surgical energy devices. Further, the laser creates extremely high temperatures in the immediate vicinity of the fiber tip (over 400° C. within 0.9 seconds, in a region of a few cubic millimeters). This requires extreme care and training on the part of the surgeon.

External ultrasound devices, e.g., ULTHERA® and LIPOSONIX® systems, depend upon direct interaction of the ultrasonic energy with tissue to cause heating. While these are non-invasive approaches, they involve energy transmission through skin and other tissues that should not be heated. Accordingly, the heating effects are designed to be very localized, on the order of one to three cubic millimeters, and delivered at a controlled rate. Because of this, these treatments are limited with regard to the area treated and the speed of treatment. Further, as the heating depends upon mechanical dissipation of the ultrasonic wave in tissue, there are other effects, such as cavitation, which must be controlled or avoided altogether.

Other devices used in skin tightening, e.g., RF and laser sources, depend upon the direct interaction of electromagnetic energy with tissue. For RF, the interaction is either Joule (resistive) heating or dielectric heating. If applied from the tissue surface, RF energy has very limited penetration, on the order of a few millimeters. Again, the heating is limited because the skin surface should not be heated. If the RF is applied internally, e.g., using a probe inserted below the skin surface, the heating can be applied more intensely. However, it can easily reach high temperatures, beyond those required to denature collagen, and thereby can cause unwanted tissue damage. Laser interaction with tissue is generally applied through optical absorption. If applied externally, laser energy penetrates to a depth that is even less than RF energy. When used internally, the temperatures at the laser fiber tip, which is on the order of 1 mm in diameter, can exceed 400° C. In this case, there can be at least a small volume of extreme tissue destruction. By moving the tip around within the tissue, an appropriate average temperature rise can be accomplished; however, there is little or no direct control over the distribution of energy within the tissue.

### SUMMARY

In one aspect, the technology relates to a surgical device comprising a probe having a proximate end configured to connect to an ultrasonic driver assembly that generates ultrasonic vibrational energy; a shaft for conducting the ultrasonic vibrational energy from the proximate end to a distal end of the probe; a canula located at least partially over the distal end of the probe; and an interposed material located between the probe and the canula for converting the ultrasonic vibrational energy into heat energy. In embodiments, the surgical device also includes heat sensors for monitoring the heat energy produced.

In another aspect, the technology comprises a canula unit that includes an outer surface adapted to distribute heat energy, an inner surface defining a lumen for receiving a probe, and one or more O-rings affixed to the inner surface, wherein the one or more O-rings comprise a viscoelastic material and are sized so as to be in contact with the probe when the probe is received.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts an embodiment of an overall layout for a tissue heating apparatus.

FIGS. 2A and 2B depict embodiments of the probe tip region of the tissue heating apparatus. FIG. 2A depicts an embodiment of the probe tip region with a viscous liquid as the interposed material. FIG. 2B depicts another embodiment of the probe tip region with a continuous viscoelastic sleeve as the interposed material.

FIG. 3 is a thermal image of an embodiment of an ultrasonically vibrated probe having three O-rings and operating at about 36 kHz frequency after about 10 seconds of excitation at an excursion of about 25 microns.

FIG. 4 is a thermal image of an outside of a canula, heated internally by an embodiment of the ultrasonically vibrated probe having three O-rings shown in FIG. 3.

FIGS. 5A-5E depict results of testing the embodiments of the ultrasonically vibrated probe shown in FIGS. 3 and 4, which demonstrate the nature of the temperature rise possible using O-rings as a viscoelastic dissipation source, including: the effect of the canula on the observed temperature rise (FIG. 5A); the effect of increasing the amplitude of the probe excursion on temperature (FIG. 5B); the effect of increasing the number of O-rings (FIG. 5C); the effect of submersing the canula in water in comparison with air (FIG. 5D); and the power applied to the generator compared to the temperature readings used for feedback in an embodiment of a probe assembly used in animal tissue (5E).

FIGS. 6A-6D depict perspective, partial tip front section, and partial tip side section views of an embodiment of a probe assembly including a hub, a probe shaft, and a probe tip.

FIGS. 7A-7C depict perspective, partial tip front section, and partial tip side section views of another embodiment of the probe assembly.

FIGS. 8A-8B depict perspective, partial tip front section, and partial tip side section views of another embodiment of the probe assembly.

FIG. 9 is a flow chart illustrating an embodiment of a method of using a surgical device for a skin tightening procedure.

### DETAILED DESCRIPTION

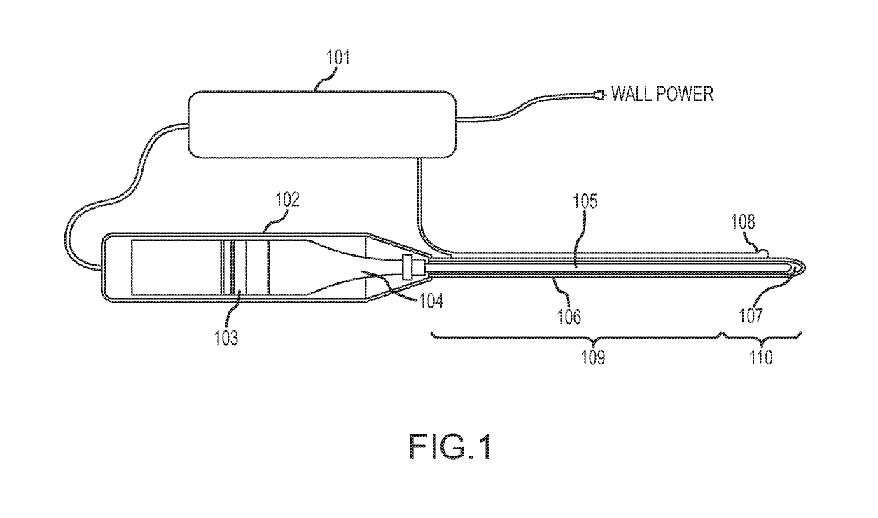

FIG. 1 is an overall diagram of a tissue heating apparatus, including a driver assembly comprising a generator 101 of high-energy electrical signals in the ultrasonic frequency range, a transducer 103 that creates ultrasonic vibrational energy in response to the electrical signals, and an ultrasonic horn (or transformer) 104 that boosts the vibrational amplitude of the transducer 103. The tissue heating apparatus further includes a housing 102 to enclose the transducer 103 and the ultrasonic horn 104, a probe 105 extending from the housing 102 and mechanically connected to the ultrasonic horn 104 to conduct the mechanical vibrational energy through a shaft region 109 to a tip region 110 of the probe 105. In embodiments, tip region 110 comprises a distal portion of the probe 105, including the probe tip and at least a distal portion of the probe shaft. The shaft region 109 comprises a proximal portion of probe 105, including a proximal portion of the probe shaft. The tissue heating apparatus further including a canula (or sheath) 106 overlying the probe 105 and the tip region 110, in close mechanical proximity to the tip region 110, and an interposed material 107 disposed between the tip region 110 of the probe 105 and the surrounding canula 106, and a means 108 to monitor the temperature of the tip region 110 for the purpose of providing feedback to control the generator 101.

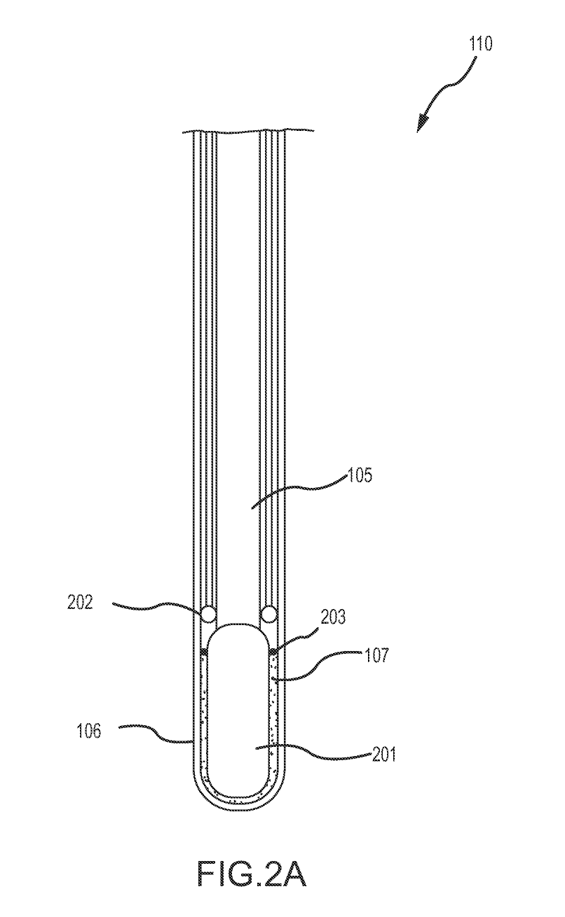

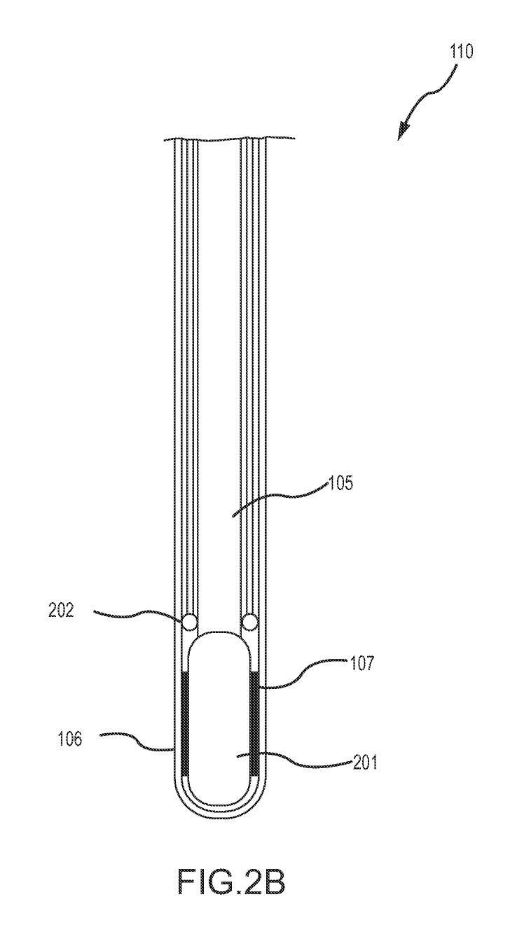

FIG. 2A depicts an embodiment of the probe tip region with a viscous liquid as the interposed material. FIG. 2A is an enlarged schematic view of the tip region 110, showing one embodiment of a probe tip 201of the probe 105, the interposed material 107 disposed between the probe tip 201 and the overlying canula 106, with temperature monitored by temperature sensors 202. In this embodiment, the interposed material 107 is a viscous liquid, with O-ring 203 creating a seal with the canula 106 to keep the viscous liquid 107 at the distal end of the probe. The temperature sensors 202 may be thermocouples (typically located on the outside of the canula, not shown) or thermistors (typically located on the inside of the canula, shown). Acceptable temperature sensors include surface mounted thermocouples, available from Quality Circuits, Inc., of Fergus Fall, Minn. and thermistors, such as those available from U.S. Sensor Corporation (www.ussensor.co), for example model KS103J2.

FIG. 2B depicts another embodiment of the probe tip region with a continuous viscoelastic sleeve as the interposed material. FIG. 2B is also an enlarged schematic view of the tip region 110, showing another embodiment of probe tip 20l of the probe 105, the interposed material 107 disposed between the probe tip 201 and the overlying canula 106, with temperature monitored by temperature sensors 202. In this embodiment, the interposed material 107 is a continuous viscoelastic sleeve disposed around the distal end of the probe. As in FIG. 2A, temperature sensors 202 are depicted as thermistors.

In embodiments, generator 101 creates high-energy electrical signals in the ultrasonic frequency range, i.e., above about 20,000 Hz. Although frequencies lower than 20,000 Hz could be used, these frequencies may have the disadvantage of creating unwanted sound in the audible frequency range. Moreover, design constraints on heating by friction or viscous losses drive the design toward higher operational frequencies. That is, a higher probe velocity results in greater frictional or viscous heating. As there is room for only a fixed displacement of the probe 105 within the overlying canula 106, higher frequencies of operation are required in order to obtain the higher probe velocities. According to embodiments, the electrical energy of the generator 101 is converted into ultrasonic vibrational energy by the transducer 103. The structures within the housing 102 and the probe 105 resonate as a unit at a specific ultrasonic frequency. The ultrasonic vibrational energy generated by transducer 103 are coupled through the ultrasonic horn 104, which boosts the vibrational amplitude of the transducer 103, and the boosted ultrasonic vibrational energy travels through probe 105 to the probe tip 201 at the distal end of probe 105. Thus, according to embodiments, the probe tip 201 vibrates with a maximum excursion because it is at the end of probe 105 and the probe 105 is part of the overall resonant structure. The probe has a pattern of vibration along its length that includes positions of minimum vibration (nodes) and maximum vibration (antinodes), and the end of the probe is at an antinode location. Depending upon the nature of the ultrasonic horn 104, the probe 105 and the probe tip 201 may vibrate either longitudinally (along the direction of the long axis of the probe 105), or circumferentially (in the “roll” direction with regard to the long axis of the probe 105). Circumferential vibration may also be referred to as “torsional vibration.”

When a vibrating element such as a probe is surrounded by a canula, it is often necessary to support the canula such that it does not directly contact the probe. This is sometimes done with O-rings placed at the node locations. For example, these O-rings are generally placed at node locations, the points of minimum vibration, so that there will be minimal frictional or other loss of vibrational energy that would create heat. Accordingly, such use of interposed material between a vibrating probe and a surrounding canula is specifically designed not to create heat.

According to present embodiments, a probe tip 201 is in intimate contact with the interposed material 107. The interposed material 107 may be a solid, a liquid, a semisolid, or some combination thereof. At least one purpose of the interposed material 107 is to convert the mechanical energy of the probe tip 201 into heat energy. This is done by pure frictional losses (i.e., by surface interaction between the interposed material 107 and an outer surface of the probe tip 201 and/or an inside surface of the canula 106), or by viscous losses (i.e., caused by relative motion between the probe tip 201 and an inside surface of the canula 106), or by viscoelastic losses (i.e., also caused by the relative motion of the probe tip 201 and an inside surface of the canula 106). The heating may also be caused by some combination of effects, depending upon the exact formulation of the interposed material 107.

For instance, one choice for the interposed material 107 would be a highly viscous liquid such as mineral oil, castor oil, or silicone oil (such as Dow Corning 710). These viscous liquids, when interposed between two surfaces (e.g., an outer surface of the probe tip 201 and an inner surface of the canula 106) that are moving rapidly relative to one another with a small distance between them, dissipate mechanical energy through viscous losses to generate heat, see, e.g., FIG. 2A. The amount of heat generated depends at least in part upon the velocity of vibration, the surface area of the probe tip 201 perpendicular to the vibration direction, and a distance between the outer surface of the probe tip 201 and the inner surface of the canula 106. The smaller the distance, the greater the viscous loss will be. According to embodiments, a sufficiently viscous liquid should be chosen in order to provide a loss mechanism. Thus, low viscosity fluids, such as water, would generally not perform adequately. On the other hand, if the fluid is too viscous and the losses are too great, there may not be sufficient energy produced by the generator 101 to drive the transducer 103. In other words, a high viscosity fluid may “load” the probe tip 201 to such an extent that the generator 101 cannot drive the load.

According to embodiments, an advantage afforded by the use of a viscous liquid is that there is substantially no abrasion or deterioration of the probe tip 201 or the viscous liquid with continued use. However, in this case, the system should be sealed in some way (such as use of an O-ring 203 on the probe 105 to create a seal with the canula 106) to keep the viscous liquid at the distal end of the probe such that heating is limited to that location. In embodiments, such sealing O-ring would be placed at a vibrational node so that it is not subject to unwanted vibration or wear.

According to other embodiments, the interposed material 107 could be a viscoelastic material such as rubber or silicone. For example, silicone O-rings made from silicone rubber (e.g., ID 0.063″×0.020″−0.024″ Cross Section, Hardness is Shore A 50 to Shore A 70) have been shown to create heat through viscoelastic and frictional losses. Suitable viscoelastic materials should exhibit viscoelastic properties as well as be able to withstand the potential heating caused by the system. In some cases, the temperature of the interposed material 107 can exceed 150° C. or more, depending upon the drive level of the probe 105 and a time duration. Unlike viscous liquids, viscoelastic materials do not present the issues of leakage or placement of the material. On the other hand, viscoelastic materials can exhibit deterioration over extended use, which could change the characteristics of the heating or perhaps stop it completely. In embodiments, if a viscoelastic material is used, it may be confined to the lateral surfaces of the probe tip 201, but not the end region 204. Additionally, in embodiments, the viscoelastic material may take the form of a continuous viscoelastic sleeve over the lateral surfaces of the probe tip 201, rather than spaced O-rings, in order to produce a more uniform heating of the canula 106. In embodiments, the spaced O-rings (or the continuous viscoelastic sleeve) are disposed in a tight mechanical connection with the vibrating probe at or near a vibrational antinode so that they absorb the ultrasonic vibrational energy and convert it to heat energy. This is in contrast to placing O-rings or other viscoelastic material at a vibrational node to provide mechanical support while minimizing heat generation.

According to still further embodiments, the interposed material 107 could be a solid, such as a ceramic or solid polymer material. An advantage of this approach is mechanical stability. However, since friction is involved, wearing of the probe tip 201 or the inside of the canula 106 may occur, depending upon which surface is subject to the relative motion of the interposed material 107. If the interposed material 107 were rigidly attached to the probe tip 201, an inner surface of the canula 106 would be the location of the frictional heat generation. Alternatively, if the interposed material 107 were rigidly attached to the canula 106, the probe tip 201 would be the location of the frictional heat generation. The choice of which surface is the source of the heating is a design choice. For instance, it is more thermally efficient to place the heating source closer to the tissue (e.g., by attaching the solid material to the probe tip 201 and thereby creating the heating source at an inner wall of canula 106), but the rigid attachment of the interposed material 107 to the probe tip 201 complicates the resonant design of the ultrasonic transducer. Alternatively, rigidly attaching the interposed material 107 to an inner wall of canula 106, thereby creating the heat source at the surface of the probe tip 201, simplifies the ultrasonic transducer design, but slows the heating of external portions of the canula 106 because the heat must travel through the interposed material to the canula 106, which is less efficient for heating of tissue.

The disclosed technology has several functional and structural distinctions over previous systems. While prior solutions (e.g., laser or RF technologies) used direct electromagnetic energy to heat tissue, the present technology uses the mechanical energy of motion at ultrasonic frequencies to create a controlled heat source for internal application. According to embodiments, since the ultrasonic vibrational energy interacts only with internal surfaces of the probe 105 and the canula 106 rather than directly with the tissue, the tissue is exposed to a controlled heat source and not the ultrasonic vibrational energy itself. Moreover, a feedback mechanism described herein maintains the temperature at a proper level to avoid tissue damage beyond that required to cause controlled collagen denaturing. The probe 105 is also larger than the laser fibers used for heating tissue and, thus, it can heat at a faster rate. Among other things, the combination of better control and faster application is an advantage over prior technologies.

In embodiments, temperature sensors (e.g. thermocouples and/or thermistors, such as thermistors 202) are used to detect the temperature of the distal end of the canula as it is being heated by the dissipative mechanisms described above. The temperature information is used to control the ultrasonic vibrational energy used to create the heating. The control mechanism provides feedback so that the temperature of the probe tip region can be maintained at a desired temperature, or target temperature. The control of the ultrasonic vibrational energy may involve changing the amplitude of the vibration, or changing the temporal pattern of the vibration (e.g. pulse width modulation or amplitude modulation). The control mechanism is typically electronic, e.g., a PID (Proportional-Integral-Derivative) control loop approach. The target temperature may be set by the operator so that it is sufficient to cause collagen shrinkage, but not so high as to cause unwanted tissue damage, i.e. tissue necrosis.

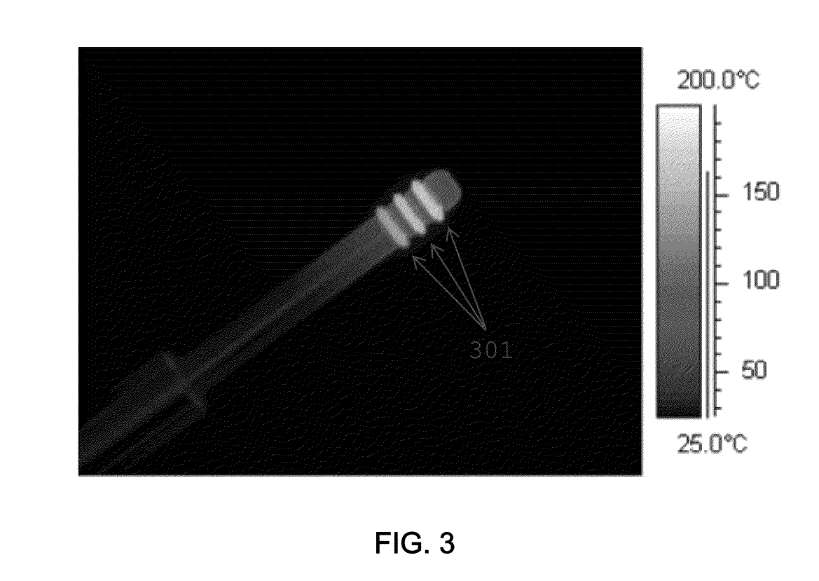

The data provided in FIGS. 3 through 5D demonstrate some of the attributes of the technology. In these exemplary embodiments, O-rings made of a viscoelastic material and spaced near the end of the probe are used; however, as discussed, other interposed materials and structures may be used to create the desired heat source.

FIG. 3 is a thermal image of an embodiment of an ultrasonically vibrated probe having three O-rings and operating at about 36 kHz frequency after about 10 seconds of excitation at an excursion of about 25 microns. The thermal image shows that the heat energy is created at the O-rings 301, which are uniformly heated between about 140° C. to about 180° C.

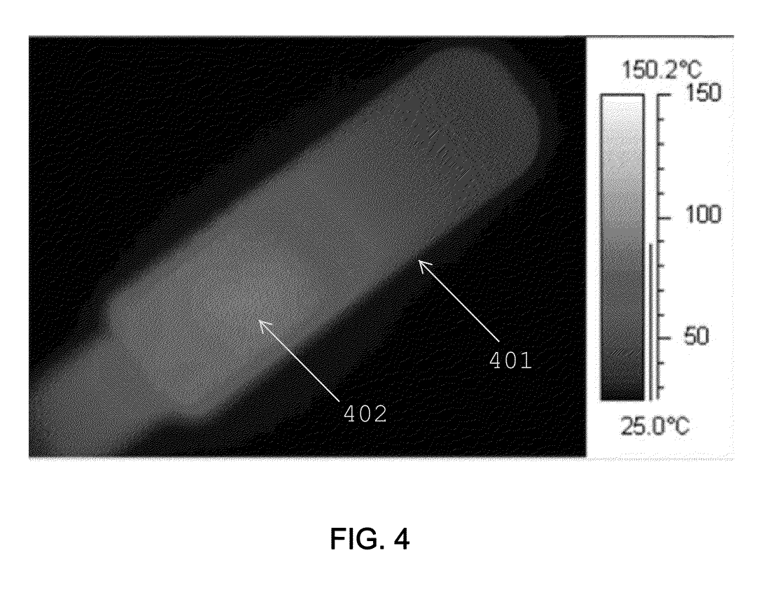

FIG. 4 is a thermal image of a canula, heated internally by an embodiment of the ultrasonically vibrated probe having three O-rings shown in FIG. 3. That is, the canula 40l has been placed over the probe with the O-rings 402. This distributes the heat generated by the O-rings substantially evenly along the outer surface of the canula 401. In this case, the temperature of the outer surface of the canula ranges between about 50° C. and about 100° C.

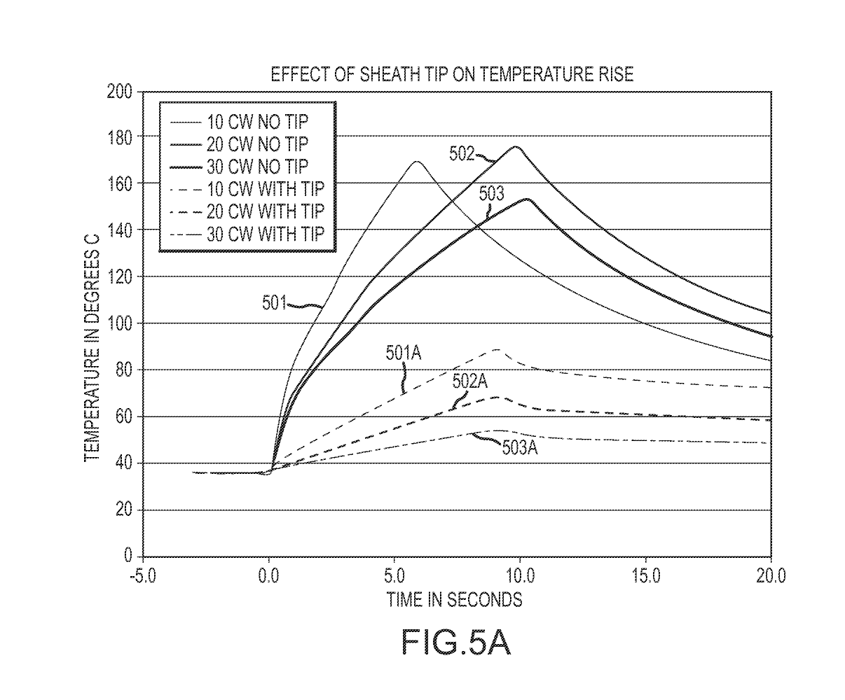

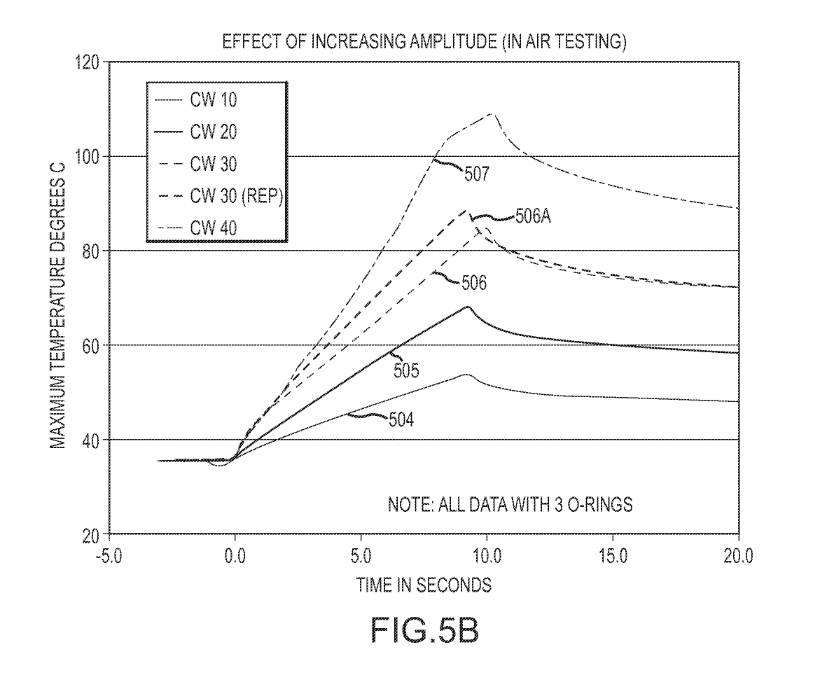

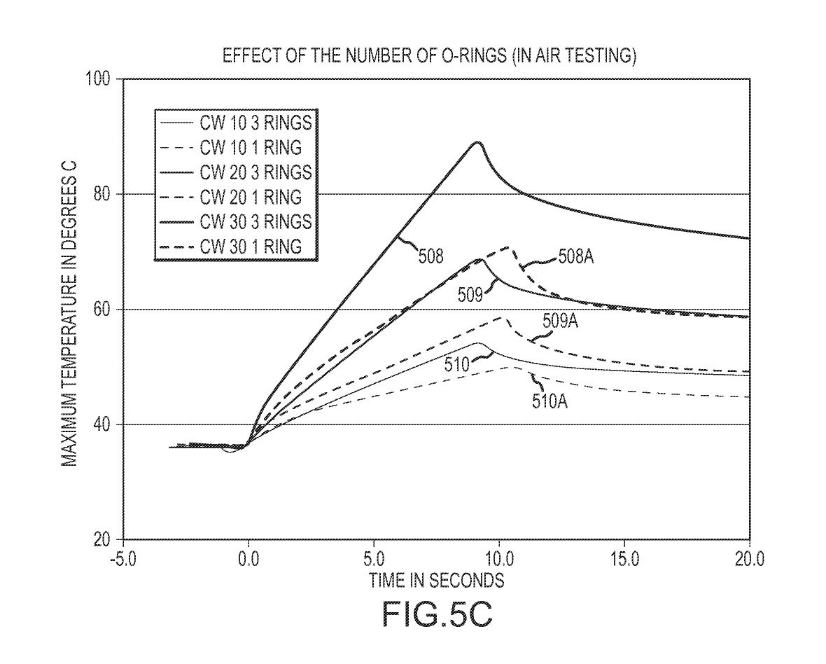

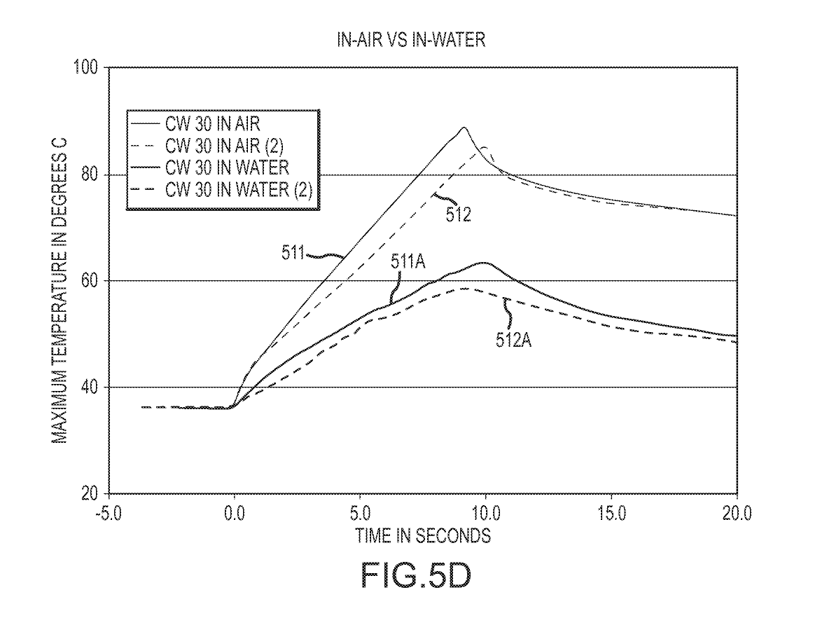

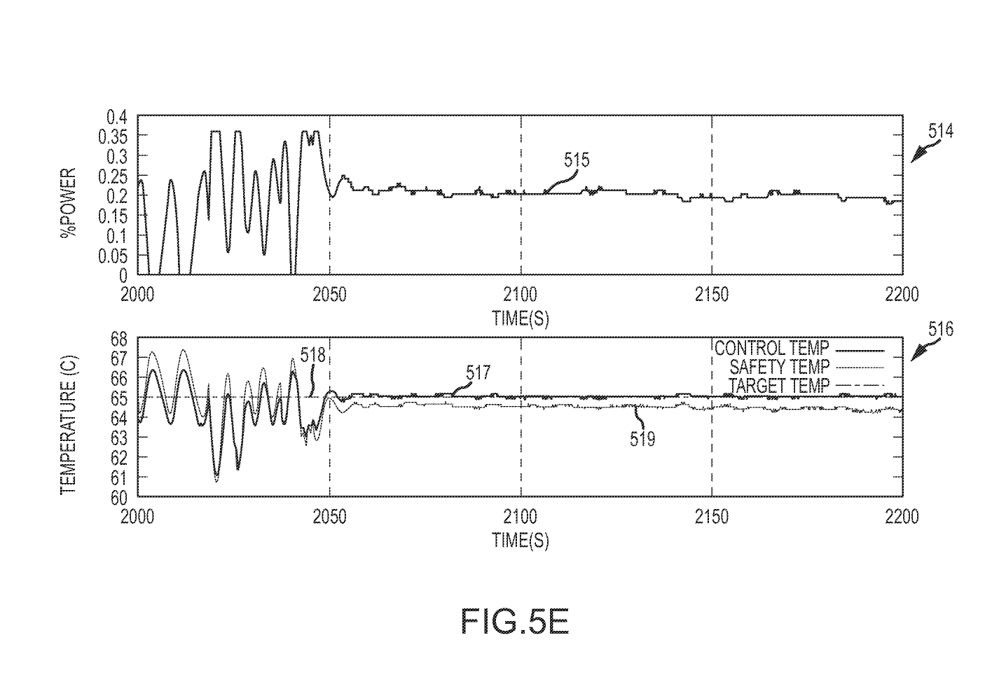

FIGS. 5A-5E depict results of testing the embodiments of the ultrasonically vibrated probe shown in FIGS. 3 and 4, which demonstrate the nature of the temperature rise possible using O-rings as a viscoelastic dissipation source. In each of these figures, the term “CW” refers to the setting on the VASER Classic device, available from Sound Surgical Technologies, LLC of Louisville, Colo., utilized in the testing procedures. In general, the CW setting increases the amplitude of the ultrasonic vibrational energy conducted by the probe.

FIG. 5A quantifies the effect of adding the canula over the probe with the O-rings, as shown in FIG. 4. More specifically, FIG. 5A shows a controlled heat rise when the canula is applied (with tip) that is lower than the heat rise of the O-rings in the absence of the canula (no tip). For example, at 30 CW, the heat rise of the O-rings (no tip) (rise 501) has a greater slope and peak than the heat rise with the canula (with tip) (rise 501A). By way of another example, at 20 CW, the heat rise of the O-rings (no tip) (rise 502) has a greater slope and peak than the heat rise with the canula (with tip) (rise 502A). In another example, at 10 CW, the heat rise of the O-rings (no tip) (rise 503) has a greater slope and peak than the heat rise with the canula (with tip) (rise 503A).

As used herein, the term slope refers to an increase in temperature over time, with a greater slope referring to a greater increase in temperature over time and a lower slope referring to a lower increase in temperature over time. The term peak refers to a maximum temperature measurement obtained within a fixed excitation time of the experiment.

FIG. 5B shows that the heat created in the O-rings without a canula corresponds to the excursion level. That is, as amplitude is increased (corresponding to a higher CW setting), there is a corresponding increase in the heat rise of the canula. For example, the heat rise over about 10 seconds at CW 10 (rise 504) has a lower slope and peak than the heat rise over about 10 seconds at CW 20 (rise 505). Similarly, the heat rise over about 10 seconds at CW 30 (rise 506 and rise 506A) has a lower slope and peak than the heat rise over about 10 seconds at CW 40 (rise 507).

FIG. 5C shows a correspondence between the number of O-rings and the heat rise of the canula. That is, as the number of O-rings is increased (e.g., from 1 to 3 O-rings), there is a proportionate increase in the heat rise of the canula. For example, the heat rise at CW 30 with 3 O-rings (rise 508) has a greater slope and peak than the heat rise at CW 30 with 1 O-ring (rise 508A). Similarly, the heat rise at CW 20 with 3 O-rings (rise 509) has a greater slope and peak than the heat rise at CW 20 with 1 O-ring (rise 509A). Further, the heat rise at CW 10 with 3 O-rings (rise 510) has a greater slope and peak than the heat rise at CW 10 with 1 O-ring (rise 510A).

FIG. 5D shows that the canula can maintain heating while submersed in a thermally dissipative fluid, in this case, water at 37° C., which simulates use of the canula in tissue. For example, the heat rise of the canula at CW 30 in air (rise 511) shows a greater slope and peak than the heat rise of the canula at CW 30 in water (rise 511A). Even so, the canula shows a controlled heat rise in water from about 37° C. to about 63° C. in about 10 seconds. Similarly, in a repeated experiment, the heat rise of the canula at CW 30 in air (rise 512) has a greater slope and peak than the heat rise of the canula at CW 30 in water (rise 512A). Again, the canula shows a controlled heat rise in water from about 37° C. to about 58° C. in about 9 seconds.

FIG. 5E depicts the power applied to a generator compared to the temperature readings used for feedback in an embodiment of a probe assembly used in animal tissue. FIG. 5E shows a portion of a data record from an experiment in which the power setting of the generator and temperature readings from two thermistors at the probe tip were recorded. The illustrated time period is from 2000 to 2200 seconds. Data chart 514 depicts the percentage power applied to the generator as the probe assembly was placed, moved around within the animal tissue, and held stationary within the animal tissue. Data chart 516 shows the temperature readings for the two thermistors. During this time period, the target temperature was set at 65° C. (reference line 518), and the probe was inserted within a piece of animal tissue that had been heated to 37° C. The data record from 2000 seconds to 2050 seconds shows changes in the power setting in response to changes in the temperature readings when the probe was moved around within the animal tissue. In embodiments, a temperature control algorithm adjusts the power setting in order to maintain the probe tip at the target temperature (target control loop). In further embodiments, there may be two thermistors, labeled “control” and “safety.” The control thermistor is used to maintain the surgical device at the target temperature, which is set by the clinician and is used to control the generator. The temperature readings measured by the control thermistor correspond to data 517. The safety thermistor is used to prevent the device from exceeding a safety temperature that is used with a separate safety circuit. The temperature readings measured by the safety thermistor correspond to data 519. The safety circuit is designed to completely shut off the generator if the safety temperature exceeds a predetermined maximum value, e.g., about 70° C. This approach provides a mechanism to shut down the system in case of failure of the target control loop. The data record from 2050 seconds to 2200 seconds shows the results while the probe was held stationary within the animal tissue. The data record between 2050 and 2200 seconds demonstrates that the control algorithm can maintain a very consistent temperature over an extended period of time.

Other experiments have demonstrated the ability of the disclosed technology to coagulate egg white, which is a substitute for collagen.

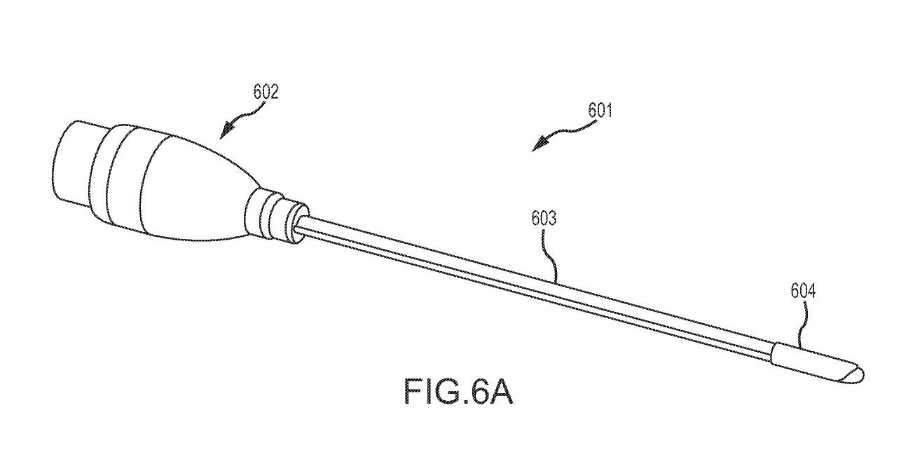

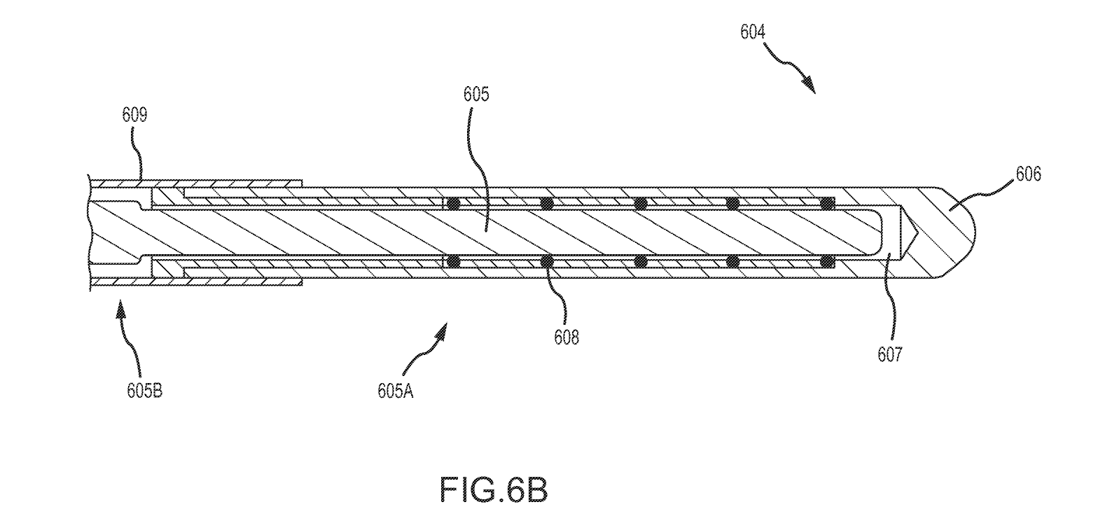

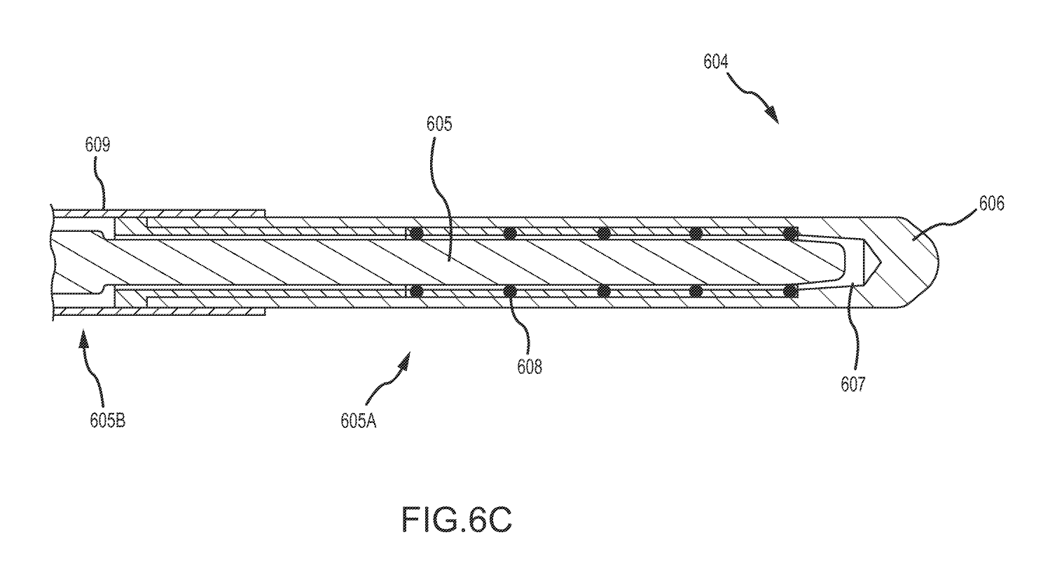

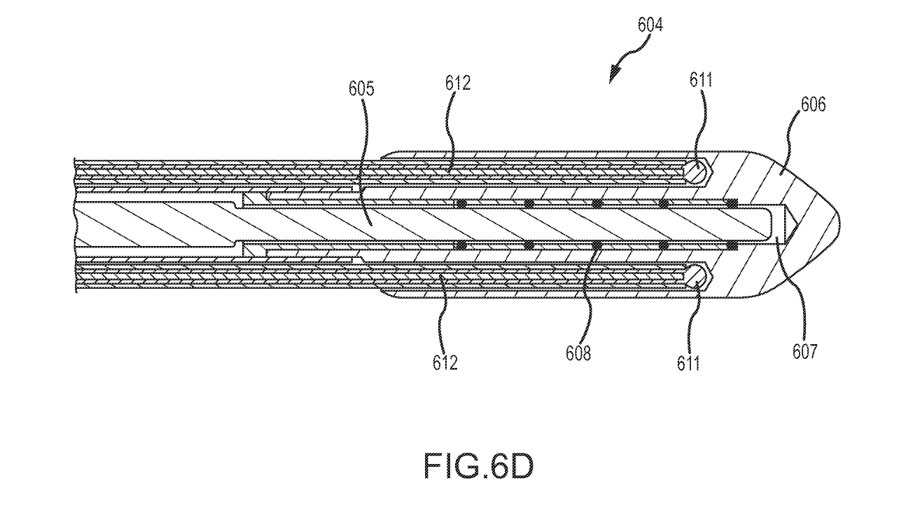

FIGS. 6A-8C depict three embodiments of probe systems. Common elements of the systems are depicted in the figures. In any of the depicted embodiments, the distal region of the probe that is in contact with the O-rings can be tapered, with the sizes of the O-rings matched to provide a uniform fit along the length of the probe distal region. This is done so that the canula can be provided to the user as a separate disposable component, since the probe is typically made from an alloy of Titanium it can be relatively expensive. By slightly tapering the probe tip, and concomitantly using O-rings of different sizes as a function of position in the canula, the probe can easily fit into the canula. Additionally, a gap within the canula lumen (between a distal end of the probe and an end of the canula lumen) may be sized to accommodate probes of different lengths. In certain embodiments, the gap may measure between 1 and 3 mm deep. According to some embodiments, the O-rings can be fitted into the canula before assembly with the probe.

FIGS. 6A-6D depict perspective, partial tip front section, and partial tip side section views of an embodiment of a probe assembly including a hub, a probe shaft, and a probe tip. More specifically, FIG. 6A shows a probe assembly 601 having a hub 602, a probe shaft 603, and a probe tip 604.

FIG. 6B shows an enlarged side section view of an embodiment of probe tip 604. Probe tip 604 comprises a distal end of probe 605 extending within canula 606. According to some embodiments, sheath 609 may overly at least a portion of probe 605 and a proximal end of canula 606 at the transition between a tip region 605A of probe 605 and a shaft region 605B of probe 605.

The interior of the canula 606 comprises a canula lumen 607 that defines a space surrounding the probe 605 such that there is not a direct mechanical connection between an outer surface of probe 605 and an inner surface of canula 606. According to embodiments, O-rings 608 are interposed between the outer surface of probe 605 and the inner surface of the canula 606 within canula lumen 607. O-rings 608 may be in close mechanical proximity to probe 605 and may form a mechanical connection between the outer surface of probe 605 and the inner surface of the canula 606 at or near vibrational antinodes so that they absorb the ultrasonic vibrational energy and convert it to heat energy. This is in contrast to placing O-rings or other viscoelastic material at a vibrational nodes to provide mechanical support while minimizing heat generation. According to embodiments, one or more O-rings 608 may be used based on suitable design, performance, or other considerations (as shown, 5 O-rings 608). Where a plurality of O-rings 608 is used, the spacing of O-rings 608 may vary based on suitable design, performance, or other considerations.

According to embodiments, O-rings 608 may be made of any suitable material that exhibits viscoelastic properties and is able to withstand the potential heating caused by the system. For example, silicone O-rings made from silicone rubber (e.g., ID 0.063″×0.020″−0.024″ Cross Section, Hardness is Shore A 50 to Shore A 70) have been found to be suitable. In embodiments, O-rings 608 convert mechanical vibrational energy of probe 605 into heat energy. For example, the O-rings 608 may create heat through frictional losses (e.g., surface interaction between the O-rings 608 and probe 605 and/or the inner surface of canula 606) and/or by viscoelastic losses (e.g., caused by the relative motion of probe 605 and an inner surface of the canula 606). The heating may also be caused by some combination of effects, depending upon the exact formulation of O-rings 608.

FIG. 6C shows an embodiment of a tapered distal region of probe 605 in which the sizes of the O-rings 608 are matched to the tapering to provide a uniform fit along the length of the probe distal region. This is done so that the canula can be provided to the user as a separate disposable component, since the probe is typically made from an alloy of Titanium it can be relatively expensive. By slightly tapering the probe tip, and concomitantly using O-rings of different sizes as a function of position in the canula, the probe can easily fit into the canula. For example, as illustrated by FIG. 6C, the distal region of the canula wall widens (and the canula lumen 607 narrows) to closely accommodate the tapered probe 605. Additionally, a gap within the canula lumen (between a distal end of the probe and an end of the canula lumen) may be sized to accommodate probes of different lengths. In some cases, the Titanium probes may vary in length from the manufacturer. In certain embodiments, the gap may measure between 1 and 3 mm deep to accommodate this variation. According to some embodiments, the O-rings can be fitted into the canula before assembly with the probe and the canula can be sold as a disposable unit. In addition, the placement of the O-rings can be predetermined to be at or near antinode positions when in contact with a particular size probe when the probe is vibrated at ultrasonic frequencies. In this manner, the O-rings may be affixed to the inner surface of the canula at or near antinode positions and sold as a prefabricated unit. In this manner the canula unit, which in embodiments may comprise the canula, O-rings, and thermal sensors, may be sold as a disposable item that can be used with the reusable portions of the surgical device (such as the probe, generator, handle, etc.

FIG. 6D shows an enlarged side section view of another embodiment of probe tip 604. Probe tip 604 comprises a distal end of probe 605 having O-rings 608 and extending within canula 606. FIG. 6D depicts Probe tip 604 further comprises two thermistors 610 located within thermistor lumens 611 that are discrete from the canula lumen 607, in which probe 605 is located. Wires 612 extend from the thermistors 610 and are substantially parallel to probe 605. In some embodiments, the wires 612 may be wrapped with sterile, medical-grade shrink wrap or another plastic element during use.

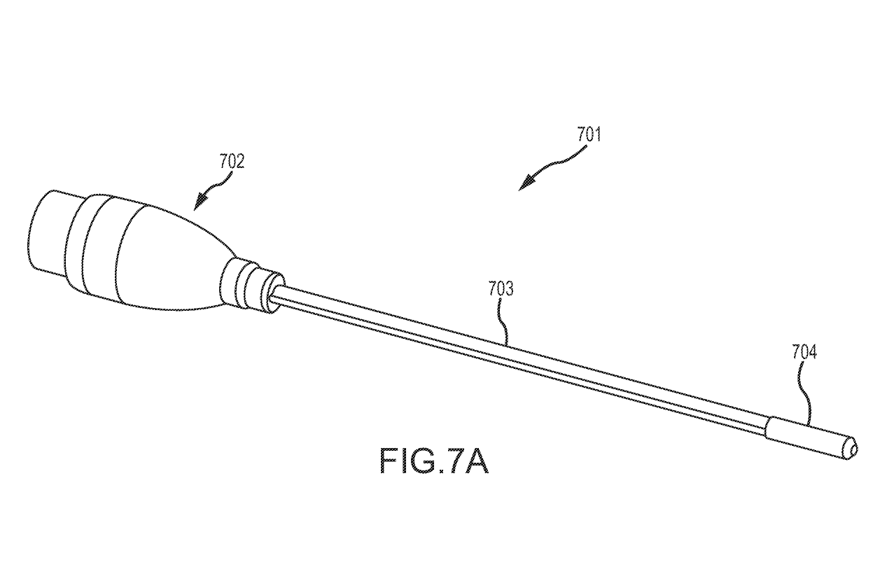

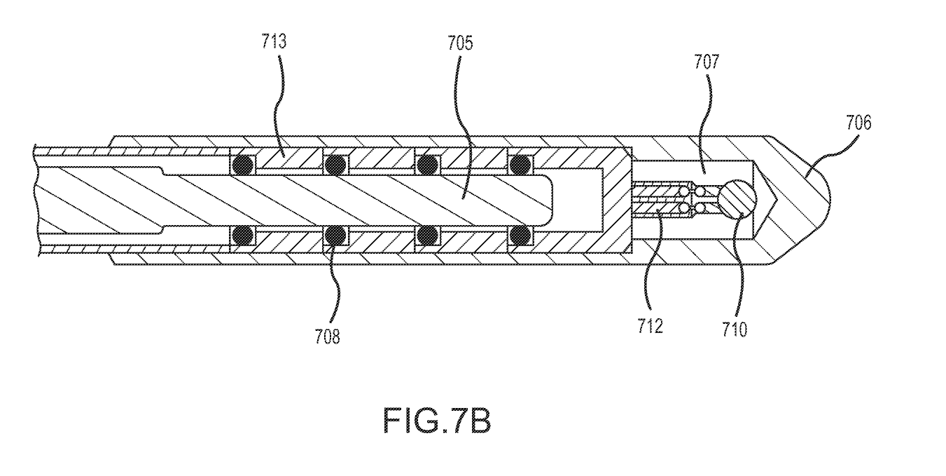

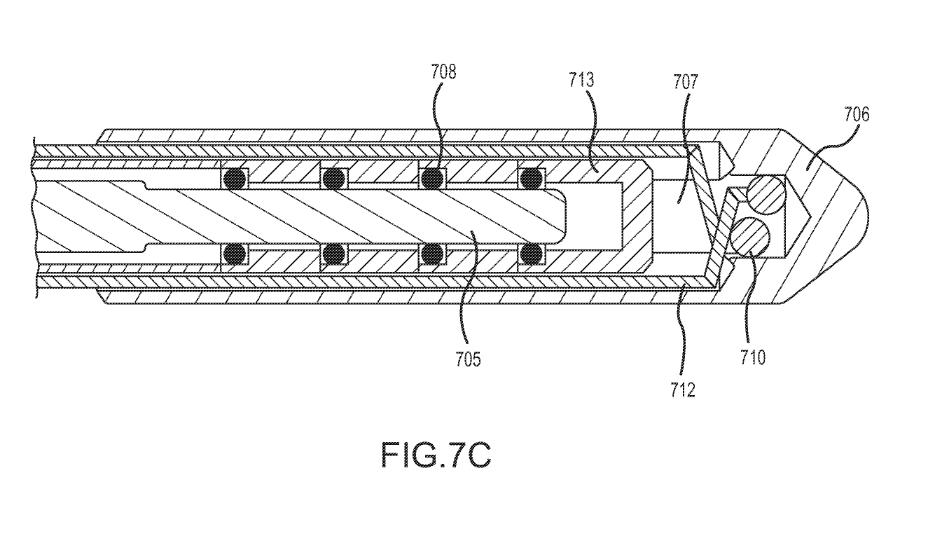

FIGS. 7A-7C depict perspective, partial tip front section, and partial tip side section views of another embodiment of the probe assembly. FIG. 7A shows a probe assembly 701 having a hub 702, a probe shaft 703, and a probe tip 704.

FIG. 7B shows a canula 706 having a central canula lumen 707 within which probe 705 having O-rings 708, thermistor 710, thermistor wires 712, and other elements are located. In this embodiment, the probe assembly includes a plurality of conductive annular elements 713 that at least partially surround the probe 705. These annular elements 713 form support surfaces for the O-rings 708, so as to fix the O-rings 708 in place relative to the probe 705.

FIG. 7C shows canula 706 having canula lumen 707 within which probe 705 having O-rings 708 is located. FIG. 7C further shows two thermistors 710 located at a distal tip of the canula lumen 707, with thermistor wires 712 thereto routed along an outside of annular elements 713. According to embodiments, one thermistor may be sufficient; however, multiple thermistors may be used for redundancy or to ensure accurate temperature readings.

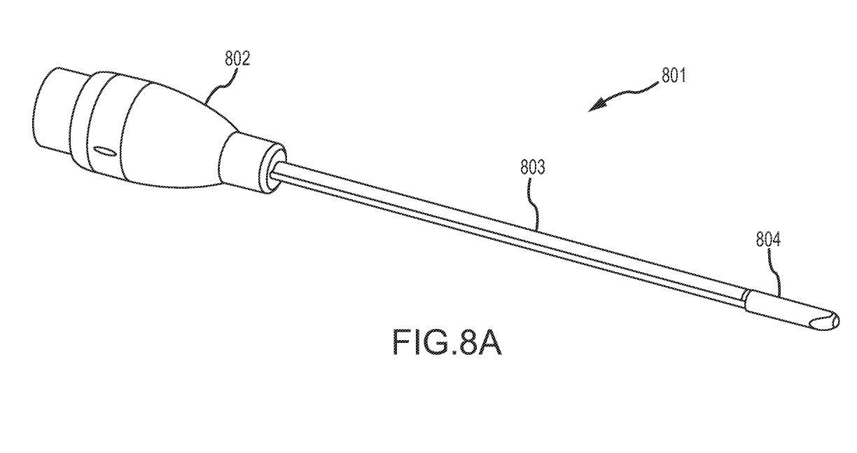

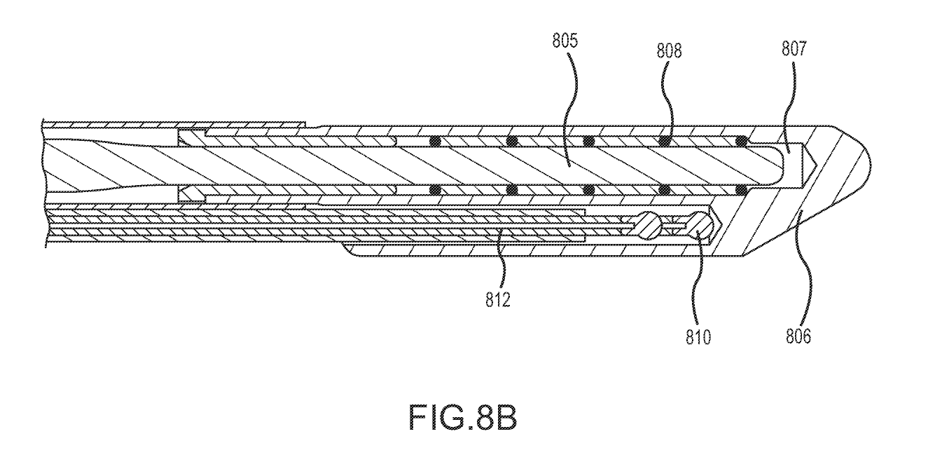

FIGS. 8A-8B depict perspective, partial tip front section, and partial tip side section views of another embodiment of the probe assembly. FIG. 8A shows a probe assembly 801 having a hub 802, a probe shaft 803, and a probe tip 804.

FIG. 8B shows two thermistors 810 proximate the distal end of the canula 806. In the illustrated embodiment, the thermistors 810 are located within a single thermistor lumen 811 within canula 806 that is discrete from canula lumen 807. The thermistor wires 812 therefrom may be held with sterile, medical-grade shrink wrap or another plastic element, such as described above.

While the preceding figures show O-rings as the viscoelastic dissipative material, a continuous viscoelastic sleeve of silicone material may be used instead (see FIG. 2B). In embodiments, the continuous viscoelastic sleeve provides a larger surface area and volume for creating dissipative heating, as opposed to discrete O-rings.

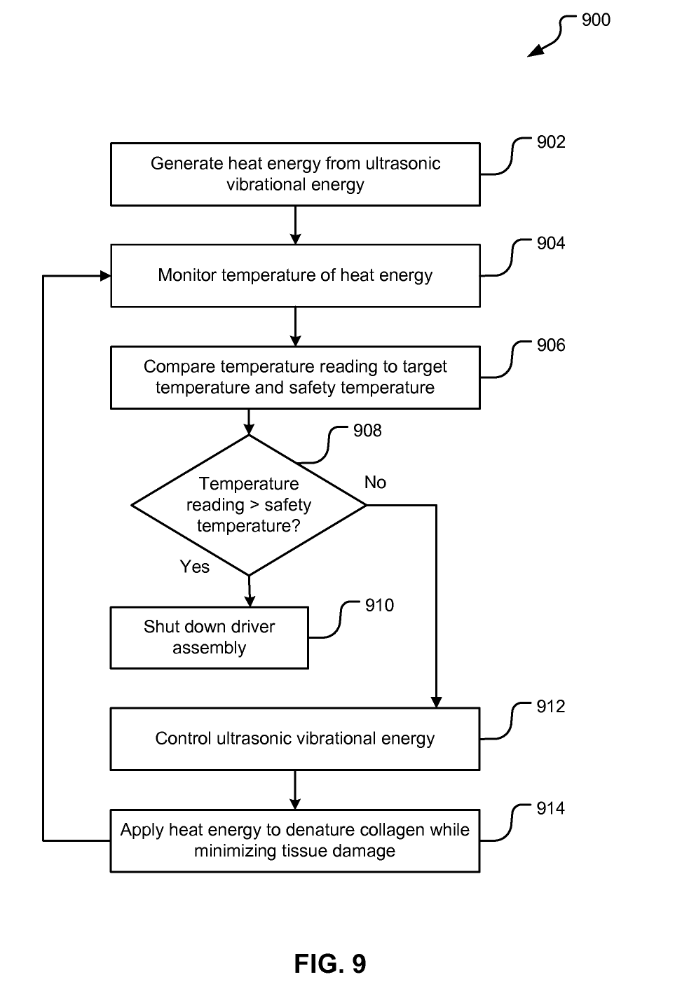

FIG. 9 is a flow chart illustrating an embodiment of a method 900 of using a surgical device for a skin tightening procedure.

At operation 902, the method comprises generating heat energy using the surgical device, the surgical device comprising a driver assembly for generating ultrasonic vibrational energy and a probe for conducting the ultrasonic vibrational energy. In embodiments, an apparatus such as the device shown and described with respect to FIGS. 6A-8B may be used to perform method 900. The probe is at least partially housed within a canula and mechanically coupled to a material interposed between an outer surface of the probe and an inner surface of the cannula, the interposed material converting the ultrasonic vibrational energy conducted by the probe into heat energy (e.g., via viscoelastic and/or frictional losses). For example, the interposed material may be in close mechanical proximity between an outer surface of the probe and an inner surface of the canula at or near a vibrational antinode so that it absorbs the ultrasonic vibrational energy and converts it to heat energy. This is in contrast to placing O-rings or other viscoelastic material at a vibrational nodes to provide mechanical support for a probe while minimizing heat generation.

According to embodiments, the ultrasonic vibrational energy is confined because the ultrasonic vibrational energy interacts directly with internal surfaces of the probe and the canula rather than directly with the tissue. Additionally, the heat energy created is controlled based on a feedback mechanism that maintains the temperature at a desired level or target temperature, e.g., between about 52° C. and 65° C. Moreover, if the heat energy exceeds a safety temperature, e.g., about 70° C., the surgical device is shut down completely. Thus, in embodiments, the heat energy of the surgical device is maintained at a target temperature not to exceed a safety temperature to avoid tissue damage beyond that required to cause controlled collagen denaturing (as described below).

At operation 904, the method comprises monitoring a temperature of the heat energy, the temperature being monitored by one or more temperature sensors. The one or more temperature sensors comprise one or more thermocouples, one or more thermistors, or some combination thereof. According to embodiments, the one or more temperature sensors may monitor the heat energy generated by the surgical device continually or periodically. For example, as described, one of the temperature sensors may be used as a control sensor to maintain the surgical device at a selected target temperature and another temperature sensor may be used as a safety sensor to prevent the surgical device from exceeding a predetermined safety temperature. The one or more temperature sensors may further continually or periodically send signal(s) representative of temperature reading(s) to the generator, or to a module communicatively coupled to the generator.

At operation 906, the method comprises comparing the temperature of the heat energy to the target temperature and the safety temperature. According to embodiments, the target temperature (or desired temperature range) is representative of a temperature (or a temperature range) at which collagen becomes denatured while damage to other tissues is minimized.

At decision operation 908, the method comprises determining whether the temperature of the heat energy is greater than the safety temperature. If the temperature of the heat energy is greater than the safety temperature, the method proceeds to operation 910. If the temperature of the heat energy is not greater than the safety temperature, the method proceeds to operation 912.

At operation 910, when the heat energy is greater than the safety temperature, the surgical device is automatically shut down.

At operation 912, when the heat energy is greater than the safety temperature, the ultrasonic vibrational energy generated by the driver assembly is controlled such that the heat energy produced by the interposed material is measured at or about the target temperature (or within the desired temperature range). According to embodiments, the ultrasonic vibrational energy generated by the driver assembly may be controlled in any suitable manner, e.g., by adjusting the amplitude or temporal characteristics of the output of the driver. According to some embodiments, the generator may further comprise a controller that is responsive to temperature feedback and is configured to control the ultrasonic vibrational energy generated by the driver assembly accordingly. For example, as illustrated by FIG. 5E, the percentage of power supplied by the driver device is strongly responsive to feedback temperature to maintain the temperature at the target temperature.

At operation 914, the method comprises applying the heat energy to denature collagen tissue in the skin of a patient while minimizing damage to other tissues. When collagen fibers are subjected to heating, their highly oriented structure is denatured or transformed into an amorphous state, with the net result of tissue shrinkage. According to embodiments, when the heat energy is maintained at a proper temperature (i.e., at or about the target temperature or within the desired temperature range), collagen is denatured while damage to other tissues is minimized. In embodiments, the method 900 or individual operations of the method 900 may represent a continuous control loop by returning to operation 904 until desired tissue shrinkage is achieved.

As should be appreciated, the particular steps and methods described herein are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present method.

Unless otherwise indicated, all numbers expressing measurements, dimensions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. Further, unless otherwise stated, the term “about” shall expressly include “exactly,” consistent with the discussions regarding ranges and numerical data. Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 4 percent to about 7 percent” should be interpreted to include not only the explicitly recited values of about 4 percent to about 7 percent, but also include individual values and sub-ranges within the indicated range. Thus, included in this numerical range are individual values such as 4.5, 5.25 and 6 and sub-ranges such as from 4-5, from 5-7, and from 5.5-6.5, etc. This same principle applies to ranges reciting only one numerical value. Furthermore, such an interpretation should apply regardless of the breadth of the range or the characteristics being described.

While there have been described herein what are to be considered exemplary and preferred embodiments of the present technology, other modifications of the technology will become apparent to those skilled in the art from the teachings herein. The particular methods of manufacture and geometries disclosed herein are exemplary in nature and are not to be considered limiting. It is therefore desired to be secured in the appended claims all such modifications as fall within the spirit and scope of the technology. Accordingly, what is desired to be secured by Letters Patent is the technology as defined and differentiated in the following claims, and all equivalents.

What is claimed is:

1. A surgical device comprising:
a probe comprising:
a proximate end configured to connect to a driver assembly that generates ultrasonic vibrational energy; and
a shaft for conducting the ultrasonic vibrational energy from the proximate end to a distal end of the probe;
a canula located at least partially over the distal end of the probe; and
an interposed material located between the probe and the canula for converting the ultrasonic vibrational energy into heat energy.

2. The surgical device of claim 1, wherein the interposed material is a viscous liquid.

3. The surgical device of claim 2, further comprising:
one or more O-rings for keeping the viscous liquid at the distal end of the probe, wherein the one or more O-rings are placed at nodal locations to minimize wear of the O-rings.

4. The surgical device of claim 1, wherein the interposed material is a continuous viscoelastic sleeve.

5. The surgical device of claim 1, wherein the interposed material comprises a viscoelastic material.

6. The surgical device of claim 5, wherein the viscoelastic material comprises one or more O-rings, wherein the O-rings are placed at or near an antinode position to generate heat energy.

7. The surgical device of claim 1, further comprising:
one or more temperature sensors for monitoring the heat energy.

8. The surgical device of claim 7, wherein the one or more temperature sensors comprise one or more thermocouples.

9. The surgical device of claim 7, wherein the one or more temperature sensors comprise one or more thermistors.

10. The surgical device of claim 9, wherein at least one thermistor is used to maintain the surgical device at a target temperature, and wherein at least one thermistor is used to prevent the surgical device from exceeding a safety temperature.

11. The surgical device of claim 7, wherein the heat energy is controlled to maintain a target temperature.

12. The surgical device of claim 11, wherein when the temperature of the heat energy reaches the target temperature, the driver assembly is adjusted to generate a different amount of ultrasonic vibrational energy.

13. A surgical device, comprising:
a probe comprising:
a proximate end configured to connect to a driver assembly that generates ultrasonic vibrational energy; and
a shaft for conducting the ultrasonic vibrational energy from the proximate end to a distal end of the probe;
a canula located at least partially over the distal end of the probe;
an interposed material between the probe and the canula, wherein the interposed material is located near an antinode position for converting the ultrasonic vibrational energy into heat energy; and
one or more temperature sensors for monitoring a temperature of the heat energy.

14. The surgical device of claim 13, wherein the interposed material is a viscous liquid.

15. The surgical device of claim 14, further comprising:
one or more O-rings for keeping the viscous liquid at the distal end of the probe.

16. The surgical device of claim 13, wherein the interposed material is a continuous viscoelastic sleeve.

17. The surgical device of claim 13, wherein the one or more temperature sensors comprise one or more thermistors, wherein at least one thermistor is used to control the surgical device at a target temperature, and wherein at least one thermistor is used to prevent the surgical device from exceeding a safety temperature.

18. The surgical device of claim 13, wherein the interposed material comprises a solid material.

19. A canula unit, comprising:
an outer surface adapted to distribute heat energy;
an inner surface defining a lumen for receiving a probe; and
one or more O-rings affixed to the inner surface, wherein the one or more O-rings comprise a viscoelastic material and are sized so as to be in contact with the probe when the probe is received.

20. The canula unit of claim 19, wherein the one or more O-rings are affixed to the inner surface such that the one or more O-rings are near an antinode position with respect to the probe when the probe is vibrated at ultrasonic frequency, and wherein the inner surface is tapered and the one or more O-rings are sized to accommodate the probe when the probe is also tapered.
